(19) United States
(12) Patent Application Publication
(10) Pub. No.: US 2021/0212438 A1
(43) Pub. Date: Jul. 15, 2021

(54) LIQUID MATERIAL IMPREGNATING MEMBER

(71) Applicant: AMG Co., Ltd., Tokyo (JP)

(72) Inventor: Hiroki HASEGAWA, Tokyo (JP)

(21) Appl. No.: 17/252,972

(22) PCT Filed: Nov. 5, 2018
(86) PCT No.: PCT/JP2018/041064

§ 371 Date: Dec. 16, 2020

(51) Int. Cl. A45D 34/04; A61K 8/02; A61Q 1/02

(52) U.S. Cl. CPC A45D 34/04; A61K 8/0208; A61K 2800/87; A45D 2200/1036; A61Q 1/02

(57) ABSTRACT

Problem to be Solved

To provide a liquid material impregnating member capable of impregnating a cosmetic composition in a shorter time.

[Solution]

A liquid substance impregnating member has a main body 10 made of foamed plastic in which open cells are formed, wherein its outer shape is defined by a side surface and an upper surface and a back surface having a larger area than the side surface; in the main body, a plurality of slits 20 and/or 30 are formed so as to be separate from each other; a plurality of slits 20 and/or 30 are distributed throughout the main body 10 and are formed so as to extend in a direction connecting the upper surface and the back surface of the main body 10; the distance between the plurality of slits 20 and/or 30 formed in the central portion of the main body 10 is smaller than the distance between the plurality of slits 20 and/or 30 formed outside the central portion; the slits 20 and/or 30 serve as pathways for the cosmetic composition to reach the open cells; and the open cells are configured to primarily function as a holding portion of the cosmetic composition.

### TECHNICAL FIELD

The present invention relates to a liquid material impregnating member for impregnation of a cosmetic composition.

### BACKGROUND ART

Conventionally, a cosmetic container for applying cosmetics has been used in which a liquid foundation is impregnated into a cosmetic composition impregnating expanded plastic foam (hereinafter referred to as a “sponge”), the sponge is arranged in a sponge holding portion of a compact-shaped cosmetic container, and the liquid foundation impregnated in the sponge is applied to a puff (for example, Patent Literature 1).

### CITATION LIST

### Patent Literature

[Patent Literature 1] JP 2017-29607

### SUMMARY OF INVENTION

### Technical Problem

In the manufacturing process of the above-mentioned cosmetic container, there is a step of impregnating the sponge with the cosmetic composition (hereinafter, referred to as “impregnation step”). As illustrated in FIG. 22, with a conventional sponge 200, for example, liquid foundation comes into contact with the outer surfaces of the main body 210 of the sponge 200 from the directions indicated by the arrows A and B, and as indicated by the arrows C1 and C2, impregnates into the inner portion of the main body 210. FIG. 23 is a schematic view illustrating an enlarged portion of the inner portion of the main body 210. The main body 210 is configured of a resin portion 212 and a cell portion 214. For example, as indicated by the arrow C1, the liquid foundation impregnates into the cell portion 214 while bending according to the internal shape of the cell portion 214. It is necessary to shorten manufacturing time through further shortening of such an impregnation step.

Based on the above, the present invention provides a liquid material impregnating member that is able to impregnate a cosmetic composition in a shorter time.

### Solution to Problem

A first invention is a liquid material impregnation member, which is a liquid material impregnating member for impregnating and holding a cosmetic composition having a foamed plastic body formed with open cells; wherein the main body has an outer shape defined by a side surface, and an upper surface and a back surface having an area greater than the side surface; in the main body, a plurality of slits are formed so as to be separate from each other; a plurality of the slits are distributed throughout the main body and are formed so as to extend in a direction connecting the upper surface and the back surface; the distance between the plurality of the slits formed in the central portion of the main body is smaller than the distance between the plurality of the slits formed outside the central portion; the slits serve as a pathway for the cosmetic composition to reach the open cells; and the open cells are configured to function primarily as a holding portion of the cosmetic composition.

According to the configuration of the first invention, the cosmetic composition comes into contact with open cells from the outer surface of the main body, passes through slits formed in the main body, and also comes into contact with open cells from the inside of the main body. The cosmetic composition that comes into contact with the open cells from the outer surface of the main body is impregnated into the open cells primarily in a direction connecting the upper surface and the back surface (hereinafter, referred to as “vertical direction”). Meanwhile, since the surfaces inside the main body formed by the slits (hereinafter referred to as “slit surface”) extend in the vertical direction, the open cells are impregnated in a direction primarily orthogonal to the vertical direction (hereinafter referred to as “horizontal direction”). In addition, since the slits are distributed throughout the main body, it is possible to generate flow of the cosmetic composition impregnated by the open cells in the vertical direction and flow of the cosmetic composition impregnated by the open cells in the horizontal direction throughout the main body. Further, since the distance between a plurality of slits formed in the central portion of the main body is smaller than the distance between a plurality of slits formed outside the central portion, the area of slit surface is relatively large in the central portion and relatively small outside the central portion. Therefore, the central portion functions as an introduction portion for a relatively large amount of the cosmetic composition to reach the open cells, and it is possible to effectively promote the impregnation of the cosmetic composition toward the outside. Through this, it becomes possible to impregnate the cosmetic composition in a shorter time.

A second invention is the liquid substance impregnating member according to the configuration of the first invention, wherein an area ratio (Sc/S1), which is the ratio of the total area (Sc) of slit surface which is the inner surface of the main body formed by the plurality of the slits to the area (S1) of the upper surface or back surface, is configured to be within a predetermined numerical range of 1 or more.

According to the configuration of the second invention, since the area of the cosmetic composition in contact with the open cells is more than doubled as compared with the case where the slits are not formed in the main body, it is possible to impregnate the liquid substance impregnating member with the cosmetic composition in an even shorter time.

A third invention is the liquid substance impregnating member according to the configuration of the first invention, wherein the slits are formed so as to pass through the main body in a vertical direction.

According to the configuration of the third invention, the cosmetic composition travels through the slits passing through the main body to impregnate the open cells of the main body.

A fourth invention is the liquid substance impregnating member according to the configuration of the first invention, wherein the slits are formed so as to be exposed on either of the upper surface or the back surface, but not be exposed on the other surface.

According to the configuration of the fourth invention, the liquid substance impregnating member is arranged in a cosmetic container so that the surface where the slits are not exposed becomes the top surface, the puff is then pressed against the top surface, it is possible to apply the cosmetic composition to the puff surface more uniformly.

A fifth invention is the liquid substance impregnating member according to the configuration of the first invention, wherein the slits are exposed on both the upper surface and the back surface, and is formed so as not to pass through the main body.

According to the configuration of the fifth invention, even if the slits are formed, since the open cell portions are not completely severed in the vertical direction, it is possible to effectively impregnate the cosmetic composition in the horizontal direction.

A sixth invention is the liquid substance impregnating member according to the configuration of the first invention, wherein a portion of the slits passes through the main body in the vertical direction; and another portion of the slits is exposed on either of the upper surface or the back surface, but not on the other surface.

A seventh invention is the liquid material impregnating member according to any one of the configurations of the first through sixth inventions, wherein the number of slits is defined as a number within a predetermined range with respect to the area of the upper surface or the back surface.

If the number of slits is too small, since the area where the cosmetic composition comes into contact with the slit surface is small, it is not possible to effectively impregnate the cosmetic composition into the open cells. Meanwhile, if the number of slits is too large, the number of positions where the open cells are severed increases, and the function of holding the cosmetic composition is impaired. In this regard, according to the configuration of the seventh invention, since the number of slits is defined as a number within a predetermined range with respect to the area of the upper surface or the back surface of the main body, it is possible to effectively impregnate as well as hold the cosmetic composition in the open cells.

An eighth invention is the liquid material impregnating member according to any one of the configuration of the first through seventh inventions, wherein the length of the slits is defined as a length within a predetermined range.

If the length of the slits is too long, a space cannot be formed between the slits. On the other hand, if the length of the slits is too short, it is not possible to secure an appropriate area of slit surface. In this regard, according to the configuration of the eighth invention, since the length of the slits is defined as a length within a predetermined range, it is possible to form spaces between the slits and secure an appropriate area of slit surface.

A ninth invention is the liquid material impregnating member according to any one of the configurations of the first through eighth inventions, wherein the slits are formed in a cross shape.

According to the configuration of the ninth invention, since the slits are formed in a cross shape, the resistance to the flow of the cosmetic composition is relatively small at the central portion in the plan view. Therefore, the cosmetic composition penetrates relatively quickly in the central portion of the slit. In addition, it is possible to increase the penetration rate of the cosmetic composition around the central portion in accordance with the higher speed of flow in the central portion.

A tenth invention is the liquid material impregnating member according to any one of the configurations of the first through ninth inventions, wherein the slits are formed based on a positional reference of a plurality of virtual concentric circles centered on a predetermined position in the central portion of the main body; and the radius of a virtual concentric circle one outside a specified virtual concentric circle of the virtual concentric circles is defined as differing only by a predetermined length from the radius of the specified virtual concentric circle of the virtual concentric circles.

### Advantageous Effect of Invention

According to the liquid substance impregnating member according to the present invention, it is possible to provide a liquid substance impregnating member capable of impregnating a cosmetic composition in a shorter time.

### BRIEF DESCRIPTION OF DRAWINGS

FIG. 1 is a schematic perspective view of a sponge according to a first embodiment of the present invention.

FIG. 2 is a schematic view illustrating the state of a sponge held in a cosmetic container.

FIG. 3 is a schematic plan view of a sponge.

FIG. 4 is a schematic plan view of a sponge with auxiliary lines.

FIG. 5 is a schematic perspective view illustrating a portion of a sponge enlarged.

FIG. 6 is a schematic view illustrating routes by which a cosmetic composition is impregnated into a sponge.

FIG. 7 is a schematic view illustrating flow of a cosmetic composition.

FIG. 8 is a schematic view illustrating horizontal flow of a cosmetic composition.

FIG. 9 is a schematic view illustrating vertical flow of a cosmetic composition.

FIG. 10 is a schematic enlarged view in a planar view of a cross-shaped slit.

FIG. 11 is a schematic view describing a permeation state of liquid in a cross-shaped slit.

FIG. 12 is a schematic view illustrating the state in which a cosmetic composition impregnates into the internal portion of a sponge.

FIG. 13 is a schematic view illustrating the state in which a cosmetic composition impregnates into the internal portion of a sponge.

FIG. 14 is a view illustrating a testing method for testing the impregnation of a cosmetic composition into a sponge and the test results.

FIG. 15 is a schematic planar view illustrating a sponge of a second embodiment.

FIG. 16 is a schematic planar view illustrating a sponge of a third embodiment.

FIG. 17 is a schematic planar view illustrating a sponge of a fourth embodiment.

FIG. 18 is a schematic bottom view illustrating a sponge of the fourth embodiment.

FIG. 19 is a schematic partial side view illustrating a sponge of the fourth embodiment.

FIG. 20 is a schematic partial side view illustrating a sponge of a fifth embodiment.

FIG. 21 is a schematic partial side view illustrating a sponge of a sixth embodiment.

FIG. 22 is a schematic view illustrating a state of the impregnation of a cosmetic composition in a conventional sponge.

FIG. 23 is a schematic view illustrating a state of the impregnation of a cosmetic composition in a conventional sponge.

### DESCRIPTION OF EMBODIMENTS

Hereinafter, preferred embodiments of the present invention will be described with reference to the drawings. The description of configurations that can be appropriately implemented by those skilled in the art will be omitted, and only basic configurations of the present invention will be described.

### First Embodiment

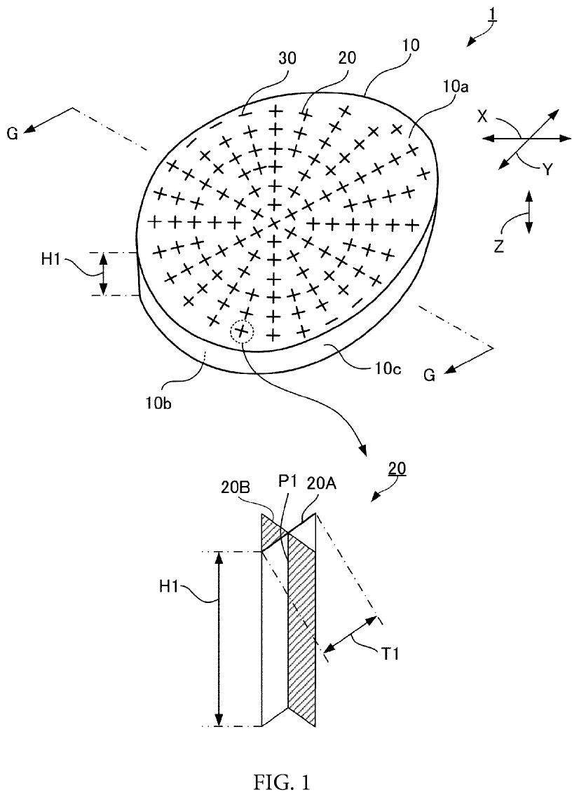

A sponge 1, illustrated in FIG. 1, is a member for impregnating and holding a cosmetic composition. The sponge 1 is an example of a liquid material impregnating member. The cosmetic composition is, for example, liquid foundation (a cosmetic foundation in liquid form). In the present specification, the cosmetic composition is described below as liquid foundation.

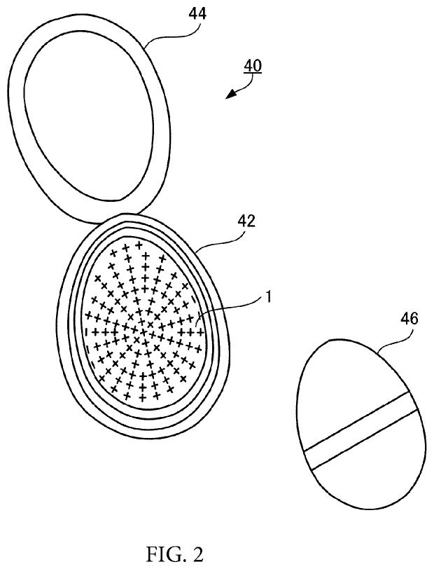

As illustrated in FIG. 2, the sponge 1 is held in a main body 42 of a compact cosmetic container 40. The cosmetic container 40 is configured such that the main body 42 and a lid 44 can be opened and closed by hinges (not shown). Liquid foundation is impregnated and held in the sponge 1. The liquid foundation held in the sponge 1 is applied to a puff 46 by the user and used for makeup.

In the manufacturing process of the cosmetic container 40, the sponge 1 is impregnated with a liquid foundation. As described below, the sponge 1 can be impregnated with the liquid foundation in a shorter time than conventionally. Through this, the manufacturing efficiency of the cosmetic container 40 can be improved.

As illustrated in FIG. 1, the sponge 1 is configured of a main body 10 which is the main body of the sponge 1. The main body 10 is formed in an overall flat shape and the outer shape is defined by an upper surface 10a, a back surface 10b, and a side surface 10c. The upper surface 10a and the back surface 10b each have a larger area than the side surface 10c. The upper surface 10a and the back surface 10b of the main body 10 have an oval shape in a plan view and have a predetermined height H1. The height H1 is, for example, 8 millimeters (mm). In the present specification, the direction connecting the upper surface 10a and the back surface 10b (the direction indicated by the arrow Z) is referred to as the vertical direction, and the directions indicated by the arrows X and Y are referred to as the horizontal direction. The vertical direction and the horizontal direction are orthogonal directions. In the present embodiment, the side surface 10c is formed perpendicular to the upper surface 10a and the back surface 10b; however, the present invention is not limited to this embodiment, and for example, the side surface 10c may also be formed as a convex curved surface from the central portion to the outside.

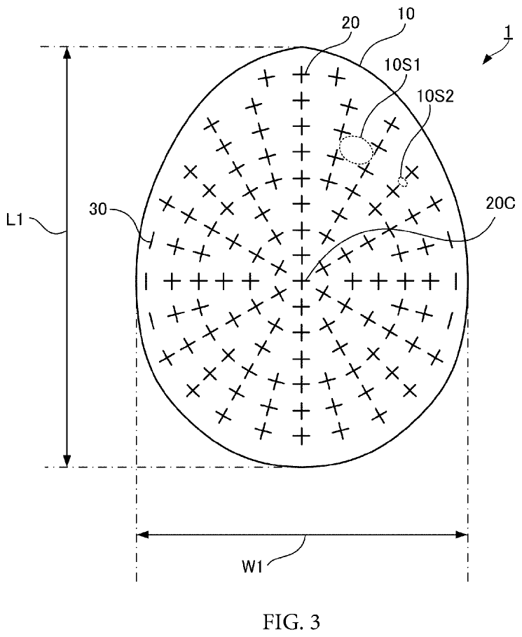

FIG. 3 is a plan view of the sponge 1. The plan view illustrates the shape of the upper surface 10a and the back surface 10b and is also a cross-sectional view taken in line G-G of the sponge 1 illustrated in FIG. 1. As illustrated in FIG. 3, the outer shape of the sponge 1 is configured of a predetermined length L1 and a width W1. The length L1 is, for example, 57 millimeters (mm). The width W1 is, for example, 45 millimeters (mm).

The main body 10 is a sponge having a large number of fine pores. The fine pores are open cells (see FIG. 5). That is, each cell of the main body 10 is not independent and is continuous with other cells. The material of the main body 10 is foamed plastic. Foamed plastic is made by foaming a synthetic resin and then solidifying it. Synthetic resins used for the foamed plastic include, for example, wet polyurethane, dry polyurethane, polypropylene, acrylonitrile-butadiene rubber (NBR), butadiene rubber (BR), styrene-butadiene rubber (SBR), natural rubber (NR), polyvinyl chloride, polyethylene, ethylene-vinyl acetate (EVA), latex, silicon, polystyrene, styrene-isoprene-styrene (SIS), styrene-ethylene-butylene-styrene (SEBS), polyvinyl alcohol (PVA), silicone elastomer, nitrile rubber, butyl rubber, polyether, polyester, and neoprene. In the present embodiment, the foamed plastic is polyethylene foam.

As illustrated in FIGS. 1 and 3, a plurality of slits 20 and 30 are formed in the main body 10 so as to be separate from each other. The slits 20 and 30 are distributed throughout the main body 10 and pass through the main body 10 in the vertical direction. The slits 20 are formed in a cross shape in a plan view, and the slits 30 are formed in a line segment shape in a plan view. Meanwhile, even in the cross-sectional view in the horizontal direction, the slits 20 have a cross shape and the slits 30 have a line segment shape.

As illustrated in FIG. 1, a slit 20 is comprised of a first portion 20A and a second portion 20B orthogonal to the first portion 20A. The first portion 20A and the second portion 20B are formed in a rectangular shape in a side view. One lengthwise direction of the first portion 20A and the second portion 20B is a direction from the central portion of the main body 10 to the outside, and the other direction is a direction orthogonal thereto. The first portion 20A and the second portion 20B have a height H1 and a width T1, respectively, and intersect at a line segment P1. A slit 30 is formed only by a first portion 20A or a second portion 20B. The width T1 is, for example, 2 millimeters (mm). As will be described later, the width T1 is defined as a length within a predetermined range.

As illustrated in FIG. 3, regions in which neither a slit 20 nor a slit 30 exists are formed in the main body 10. In the main body 10, a region in which neither a slit 20 nor a slit 30 is formed is referred to as a “base portion”. A base portion of the main body 10 is, for example, a base portion 1051 surrounded by four slits 20 or a base portion 1052 between two slits 20. Liquid foundation is impregnated and held in base portions such as the base portions 1051 and 1052. More specifically, as will be described later, liquid foundation is impregnated and held in the open cells of the base portions.

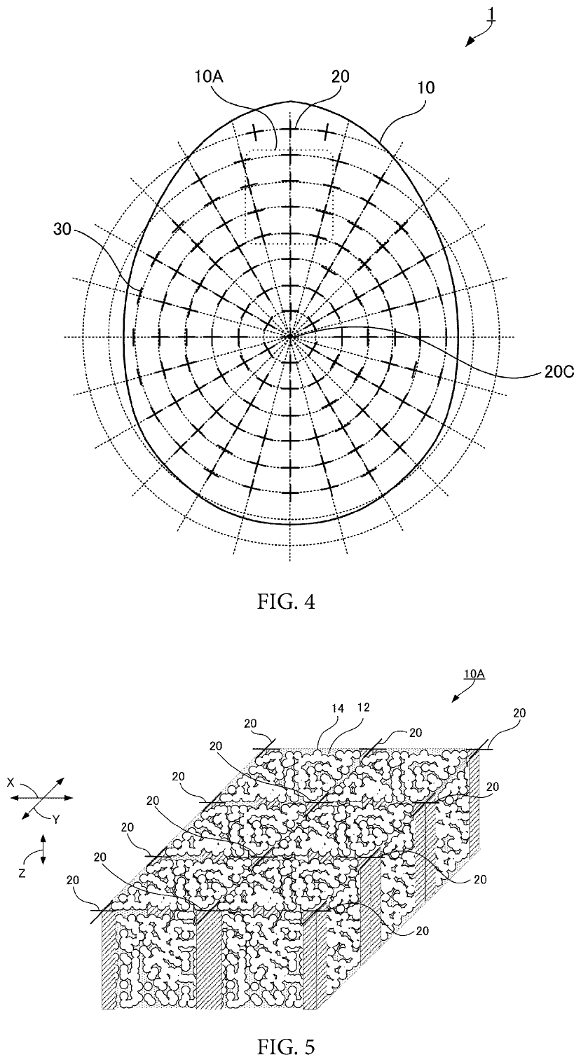

As illustrated in FIGS. 1 and 3, the distance between a plurality of slits 20 formed in the central portion of the main body 10 is smaller than the distance between a plurality of slits 20 and 30 formed in portions outside of the central portion. Hereinafter, the arrangement of the slits 20 and 30 will be described in detail while referencing FIG. 4.

As illustrated in FIG. 4, the slits 20 and 30 are formed based on the position of a plurality of virtual concentric circles centered on a predetermined position in a central portion of the main body 10. In FIG. 4, the slit 20 at the center of the central portion of the main body 10 is shown as a slit 20C. The radius of the virtual concentric circle one outside a specific virtual concentric circle is defined to be different by a predetermined length in comparison with the radius of the specific virtual concentric circle. For example, the radius of the most central virtual concentric circle is 7 millimeters (mm), the radius of a virtual concentric circle one toward the outside thereof is 14 millimeters (mm), and the radius of a virtual concentric circle one further toward the outside thereof is 21 millimeters (mm). That is, the radius of the virtual concentric circles increases by a predetermined length such as 7 millimeters (mm) with each progressive one toward the outside. In principle, the same number of slits 20 or 30 are arranged along each virtual concentric circle. Therefore, the number of slits 20 and 30 per unit of area is greater toward the center and less toward the outside.

The number of slits 20 and 30 is defined as a number within a predetermined range with respect to the area of the upper surface 10a or the back surface 10b of the main body 10. The surface within the main body 10 formed by the slits 20 is referred to as a “slit surface”. If the number of slits 20 is too small for the main body 10 as a whole, since the area where the liquid foundation comes into contact with the slit surface is small, the liquid foundation is not able to be effectively impregnated into the open cells. Meanwhile, if the number of slits 20 is too large, the number of positions where the open cells are severed increases, and the overall function of holding the liquid foundation of the main body 10 is impaired. In this regard, since the number of slits 20 is defined as a number within a predetermined range with respect to the area of the upper surface 10a or the back surface 10b of the main body 10, the liquid foundation is able to be effectively impregnated and held in the open cells.

The width T1 of the slits 20 and 30 is defined as in a predetermined numerical range in relation to the width W1 of the main body 10. The predetermined numerical range has the ratio A1 (T1/W1) of the width T1 and the width W1 as 0.03 or greater and 0.06 or less. In the present embodiment, since the width T1 of the slits 20 and 30 is 2 millimeters (mm) and the width W1 of the main body 10 is 45 millimeters (mm), the ratio A1 is 0.04. If the length of the slits 20 is too long, a space cannot be formed between the slits 20. Meanwhile, if the length of the slits 20 is too short, an appropriate area of slit surface is not able to be secured. In this regard, since the length of the slits 20 is defined as a length within a predetermined range, it is possible to form a space between the slits 20 as well as secure an appropriate area of slit surface.

The slits 20 whose width T1 is defined as described above are defined to be four or more and seven or less per unit of area (1 square centimeter: 1 cm²) of the main body 10 in a plan view (that is, upper surface 10a and back surface 10b), and preferably five to six. In the present embodiment, for example, the number is about five per unit of area. The number of slits 30 is defined as 10% or less the number of slits 20. In the present embodiment, for example, the number of slits 20 is 105 and the number of slits 30 is 6, so the number of slits 30 is 5.7% the number of slits 20. If the number of slits 20 is too small, it is not possible to secure an appropriate area of slit surface. Meanwhile, if the number of slits 20 is too large, the position where the open cells are severed is too large, and the function of holding the liquid foundation is impaired. In this regard, since the number of slits 20 is defined as a number within a predetermined range, it is possible to secure the liquid holding function of open cells while securing an appropriate area of the slit surface.

As described above, a slit 20 is configured of a first portion 20A and a second portion 20B, and a slit 30 is configured of a first portion 20A or a second portion 20B. Therefore, the total number of first portions 20A and the second portions 20B configuring the slits 20 is 210, and the total number of first portions 20A or second portion 20B configuring the slits 30 is six. The total number of first portions 20A and second portions 20B configuring slits 20 and 30 is 216. The total number of first portions 20A and second portions 20B is defined to be eight or more and 14 or less per unit area (1 square centimeter: 1 cm²) of the main body 10 in a plan view, and preferably 10 or more and 12 or less. In the present embodiment, the number is about 11 per unit area.

By forming the slits 20 and 30 as described above, the area ratio (Sc/S1) of the total area Sc of the slit surface to the area S1 of the upper surface 10a or the back surface 10b is configured so as to be within a predetermined numerical range of 1 or more. This makes it possible to form a large area where the liquid foundation is in contact with the open cells in the impregnation step. The area ratio (Sc/S1) is preferably 1 or more, for example, 1.0 or more and 2.5 or less, and preferably 1.5 or more and 2.5 or less. Focusing on one slit 20 or 30, within the main body 10, while slit surfaces are formed on both sides of one slit 20 or 30 (for example, surfaces 20Aa and 20Ab in FIGS. 10 and 11), in calculating the area ratio (Sc/S1) of the total area Sc, the area of one surface is used (for example, the surface 20Aa among the surfaces 20Aa and 20Ab of FIGS. 10 and 11). Unlike with the present embodiment, when the area of both sides of one slit 20 or 30 is used, the area ratio (Sc/S1) becomes twice that of the case of using the area of one side, but technically is synonymous with the present embodiment.

Since the area S2 of the first portion 20A or the second portion 20B is respectively S2=T1×H1, with a width T1 of 0.2 centimeters (cm) and a height H1 of 0.8 centimeters (cm), the area S2 is 0.16 square centimeters (cm²). In addition, since the total number of the first portions 20A or the second portion 20B configuring the slits 20 and 30 is 216, the total area Sc is 34 square centimeters (cm²). Assuming that the area S1 is about 20 square centimeters (cm²), the area ratio (Sc/S1) is 1.7.

FIG. 5 is a diagram conceptually illustrating an enlarged region 10A of the main body 10 of the sponge 1 illustrated in FIG. 4. As illustrated in FIG. 5, the base portion of the region 10A is configured of a resin portion 12 as a base material and a cell portion 14 formed by foaming the resin portion 12. The individual cells configuring the cell portions 14 are continuous with the other cells to form open cells. The liquid foundation is impregnated and held in the cell portions 14. Slits 20 are formed so as to surround the base portion.

The cell portions 14 have continuous spherical spaces and a non-linear complex shape, and mainly function as liquid foundation holding portions. Meanwhile, the slits 20 have relatively low resistance to the liquid foundation flow, and function as pathways for the liquid foundation to reach the cell portions 14.

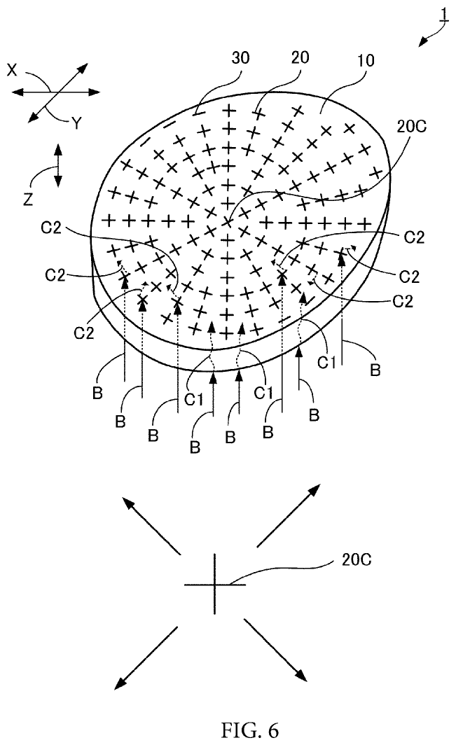

In the step of impregnating the sponge 1 with the liquid foundation, for example, as illustrated in FIG. 6, the liquid foundation comes into contact with the main body 10 in the direction from the bottom toward the top as shown by the arrows B. In FIG. 6, for convenience of explanation, only the arrows B from the bottom toward the top are shown, but in the impregnation step, the liquid foundation comes into contact with the main body 10 from any direction on the outer surface. For example, the main body 10 may come into contact from the top toward the bottom, or the main body 10 may come into contact from the side.

The liquid foundation in contact with the base portions of the main body 10 is impregnated, for example as indicated by arrows C1 in FIGS. 6 and 7, from the bottom upward through the cell portions 14 (see FIG. 5) bending along the shape of the cell portions 14. Meanwhile, the liquid foundation in contact with the slits 20 or 30 penetrates the slits 20 and 30 which have a relatively low flow resistance compared to the base portion and is impregnated while bending in a horizontal direction as indicated by the arrows C2 illustrated in FIGS. 6 and 7.

In the present specification, “penetration” is used to mean that a liquid passes through a gap in an object, and “impregnation” is used to mean that it soaks into a gap in a construction or structure. In addition, regarding the speed of liquid flow, the speed of penetration is faster than the speed of impregnation. In the present specification, regarding the direction of liquid flow, the direction from the bottom upward or vice versa (that is, the vertical direction) does not necessarily mean a linear direction, but also includes a bending direction. Further, regarding the direction of liquid flow, the horizontal direction does not necessarily mean a linear direction, but also includes a bending direction. That is, in the present specification, regarding the directions of liquid flow, “vertical direction” and “horizontal direction” are used as relative concepts for distinguishing each other as different directions.

As described above, in the plan view, since the number of slits 20 and 30 per unit area is greater toward the center and less toward the outside, in the central portion, a relatively large amount of liquid foundation penetrates the slits 20 and 30 in the vertical direction. In the process of penetration, the liquid foundation comes into contact with the cell portions 14 (see FIG. 5) and impregnates the cell portions 14 in the horizontal direction. When illustrated conceptually, as illustrated in FIGS. 6 and 7, the liquid foundation impregnates the cell portions 14 in the horizontal direction radially from the slit 20C in the central portion.

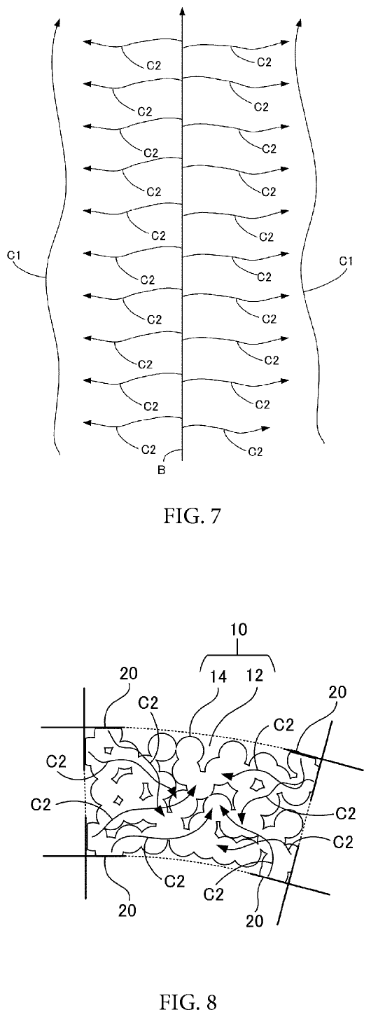

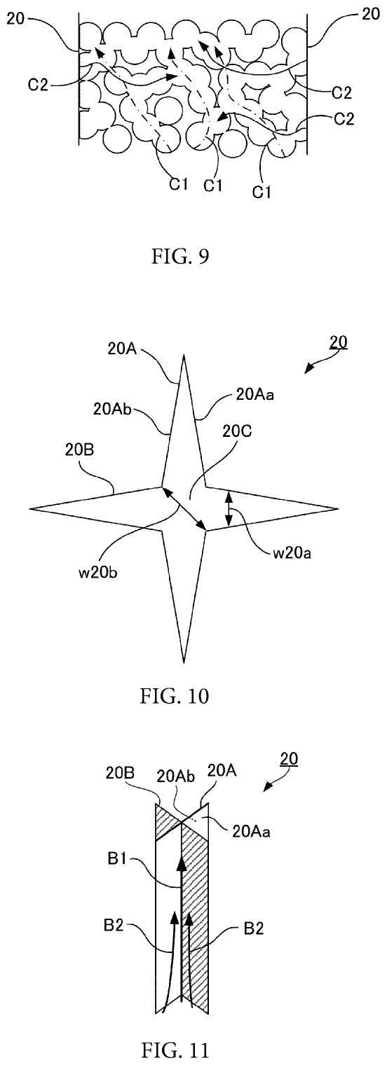

As illustrated in FIGS. 8 and 9, the liquid foundation that has penetrated into the slits 20 impregnates the cell portion 14 in the horizontal direction as indicated by the arrows C2. Further, as illustrated in FIG. 9, the liquid foundation directly in contact with the cell portions 14 from the outer surface of the base portion of the main body 10 impregnates the cell portions 14 through the flow from the bottom upward indicated by the arrows C1.

The configuration of a slit 20 will be described while referencing FIG. 10. As described above, the slit 20 is configured of a first portion 20A and a second portion 20B. Since the slit 20 is a notch, there is no concept of the width of the slit, but when liquid penetrates, a slight gap develops. As illustrated in FIG. 10, when the liquid penetrates, the maximum width w20b of a gap in the central portion is larger than the width w20a of a gap other than in the central portion. Accordingly, at the central portion of the slit 20, resistance to the flow of the liquid foundation is relatively low.

Therefore, as illustrated in FIG. 11, the flow B1 of the liquid foundation in the central portion of the slit 20 develops. In addition, the flow B1 promotes the flow B2 of the liquid foundation surrounding the central portion. This allows the liquid foundation to penetrate the slit 20 more effectively.

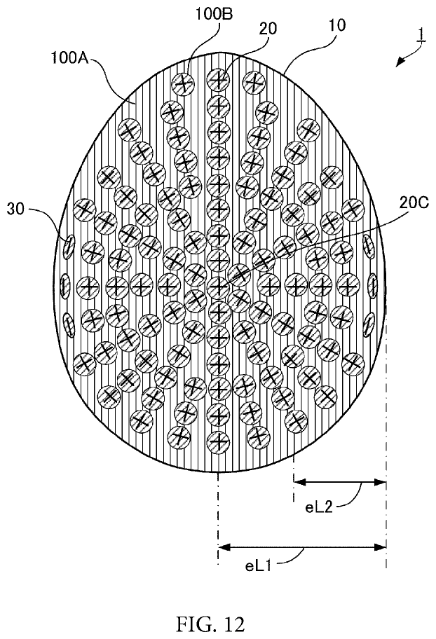

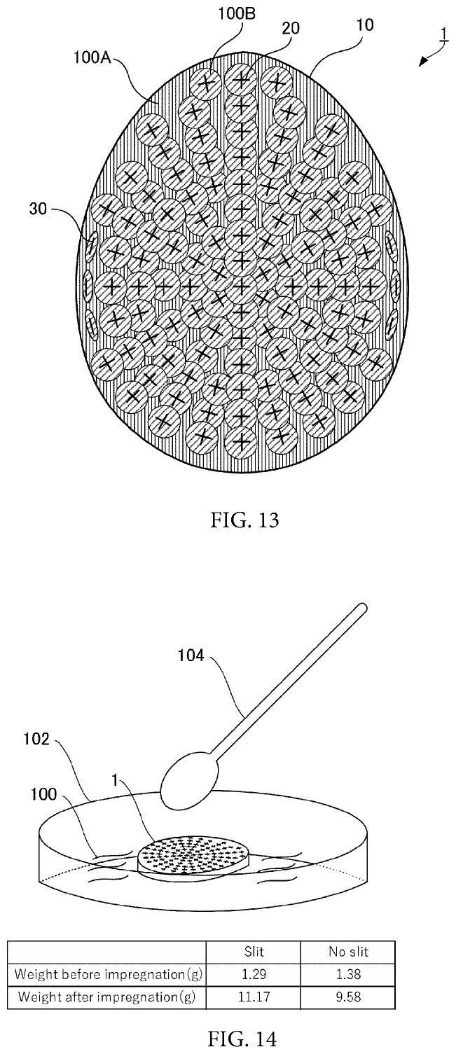

As illustrated in FIG. 12, the sponge 1 is configured so that when impregnating the liquid foundation, there develops a liquid foundation 100A which directly comes into contact with the cell portions 14 and impregnates from the outer surface of the base portion of the main body 10, and a liquid foundation 100B which impregnates by penetrating slits 20 and 30 and then coming into contact with the cell portions 14. As time passes from the state of FIG. 12, as illustrated in FIG. 13, the amounts of the liquid foundations 100A and 100B impregnating the sponge 1 increase.

The liquid foundation 100A impregnates the entire main body 10 almost uniformly. Meanwhile, the liquid foundation 100B penetrates a relatively large amount in the central portion of the main body 10 and impregnates outward.

If the configuration of the sponge 1 is different from that of the present embodiment and the number of slits 20 and 30 per unit area is smaller toward the central portion and larger toward the outer portion in a plan view, the cell portions 14 are unable to be effectively impregnated. This is because the distance from the outer portion to the end of the main body 10 (for example, the distance eL2 in FIG. 12) is shorter than the distance from the central portion to the end of the main body 10 (distance eL1), and the volume of the cell portions 14 from the outer portion to the end of main body 10 is relatively small. Meanwhile, in the sponge 1 of the present embodiment, since the number of slits 20 and 30 per unit area is greater in the central portion and less toward the outer portion in a plan view, a relatively large amount of liquid foundation 100B penetrates the central portion and effectively impregnates a relatively large volume of cell portions 14 between the central portion and the end of the main body 10.

FIG. 14 is a diagram illustrating a testing method and testing results for impregnating the sponge 1 with liquid foundation. The liquid foundation 100 is added to a petri dish 102 and the sponge 1 is set to soak. The amount of the liquid foundation 100 is adjusted so that the sponge 1 can be submerged in the liquid foundation 100. Next, the sponge 1 is pressed 30 times within a predetermined time with a laboratory spoon 104. The components of the liquid foundation are 22% water, 15% cyclopentasiloxane, 11% triethylhexanoin at mass percent concentration (weight percent concentration) (wt %); other components thereof include PG, Diphenylsiloxy Phenyl Trimethicone, trimethylsiloxysilicate, PEG-10 dimethicone, ethylhexyl methoxycinnamate, hydroxypropyl cyclodextrin, isotridecyl isononanoate, ethanol; and the viscosity is 10,000 mPa·s (millipascal seconds: Type B viscometer, No. 4 rotor, 12 rpm, 20° C.)

This testing was carried out under the same conditions for both the sponge 1 having the slits 20 and 30 formed and a comparison sponge having no slits 20 or 30 formed. The difference between the sponge 1 and the comparison sponge is the presence or absence of slits 20 and 30; the materials and shapes are substantially the same. In FIG. 14, the description of “Slit” is the sponge 1, and the description of “No Slit” is the comparison sponge. “Weight before impregnation” is the pre-impregnation weight, and “Weight after impregnation” is the post-impregnation weight. The difference between the post-impregnation weight and the pre-impregnation weight is the impregnation content (mass) of the impregnated liquid foundation 100.

Since the impregnation content of the sponge 1 is 9.88 grams (g) (11.17-1.29) and the pre-impregnation content is 1.29 grams (g), the impregnation content per pre-impregnation gram (g) (hereinafter referred to as “unit impregnation content”) is 7.66 grams (g). Meanwhile, since the impregnation content of the comparison sponge is 8.2 grams (g) (9.58-1.38) and the pre-impregnation weight before impregnation is 1.38 g (g), the unit impregnation content is 5.94 grams (g). When compared by unit impregnation content, the sponge 1 is 1.72 grams (g) higher. 1.72 grams (g) corresponds to 29 percent (%) of the 5.94 gram (g) unit impregnation content of the comparison sponge. That is, by forming the slits 20 and 30 having the above-described configuration, the sponge 1 has a unit impregnation content increased by 29% as compared with the case where the slits 20 or 30 are not formed. This means that with the same amount of time, the unit impregnation content increased by 29%, and means that the sponge 1 was able to impregnate more liquid foundation in a shorter time.

In the sponge 1, since the open cells are severed by forming the slits 20 and 30, the amount of liquid foundation that can be held in the open cells is reduced. Nevertheless, the fact that the sponge 1 impregnated a greater amount of liquid foundation than the comparison sponge according to the above testing means that the slits 20 and 30 have a significant effect of increased area of the liquid foundation coming into contact with the open cells.

### Second Embodiment

A second embodiment will be described focusing on portions differing from the first embodiment. As illustrated in FIG. 15, in a sponge 1A of the second embodiment, except for the cross-shaped slit 20 formed in the central portion, line segment-shaped slits 30 are formed.

### Third Embodiment

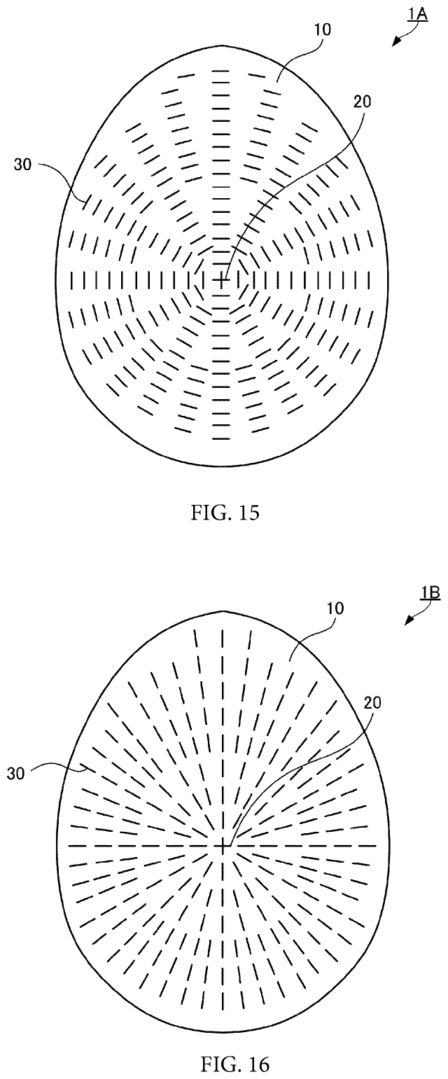

A third embodiment will be described focusing on portions differing from the first embodiment. As illustrated in FIG. 16, in a sponge 1B of the third embodiment, except for the cross-shaped slit 20 formed in the central portion, line segment-shaped slits 30 are formed. The line segment direction of the slits 30 is formed pointing toward the central portion.

### Fourth Embodiment

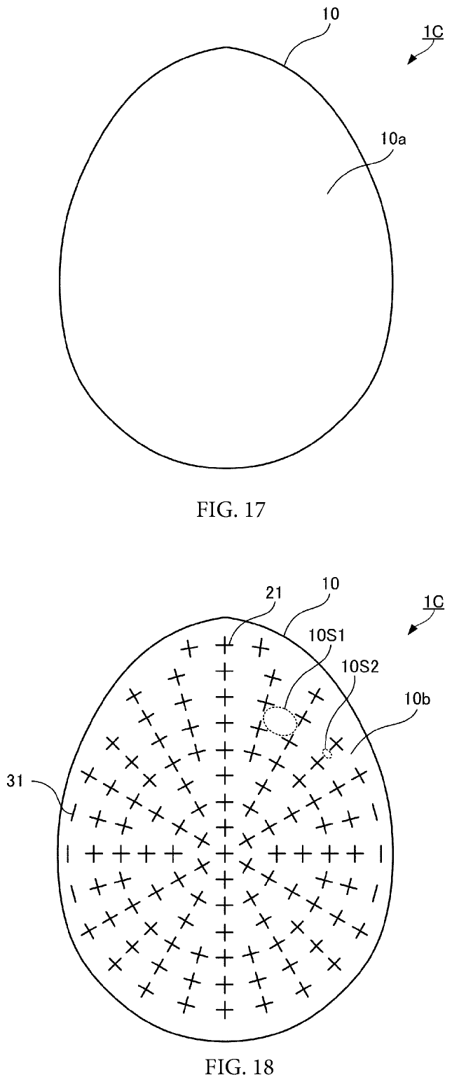

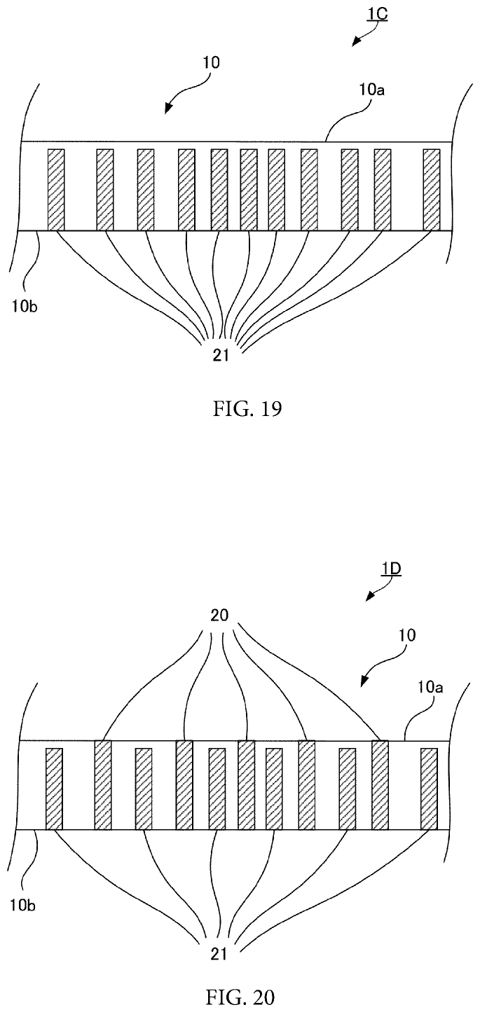

A fourth embodiment will be described focusing on portions differing from the first embodiment. As illustrated in FIGS. 17 to 19, in a sponge 1C of the fourth embodiment, the slit 21 and the slit 31 are formed in the main body 10, but the slits 21 and the slits 31 do not pass through the main body 10 in the vertical direction. The slits 21 and the slits 31 are exposed on a back surface 10b (see FIG. 18), but the slits 21 and the slits 31 are not exposed on an upper surface 10a (see FIG. 17). The sponge 1C is disposed in the cosmetic container 40 so that the upper surface 10a with no exposed slits 21 and slits 31 is exposed. As a result, when the user presses the puff 46 against the sponge 1C, the puff 46 comes into contact with the upper surface 10a where the slits 21 and the slits 31 are not exposed, so that the liquid foundation can be more evenly applied to the surface of the puff 46.

### Fifth Embodiment

A fifth embodiment will be described focusing on portions differing from the fourth embodiment. As illustrated in FIG. 20, in a sponge 1D of the fifth embodiment, slits 21 that do not pass through the main body 10 in the vertical direction and slits 20 that do pass through the main body 10 are formed. As a result, the effect with the first embodiment and the effect with the fourth embodiment are both achieved.

### Sixth Embodiment

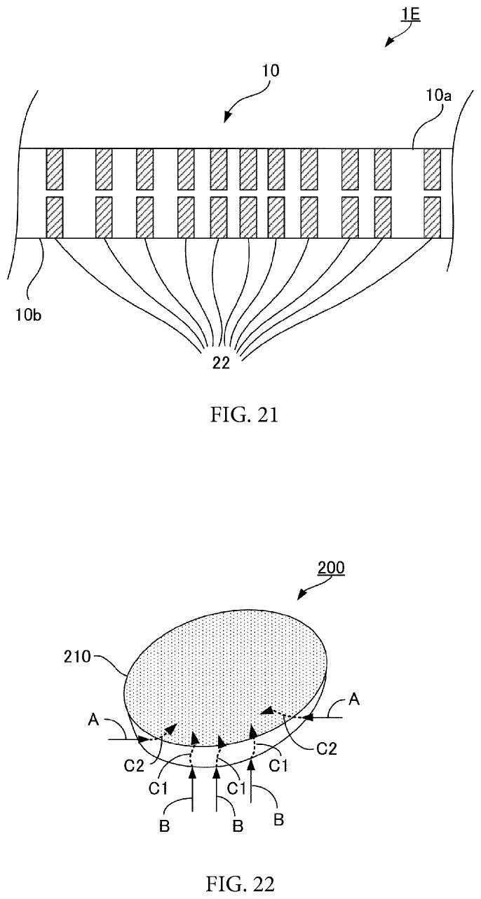

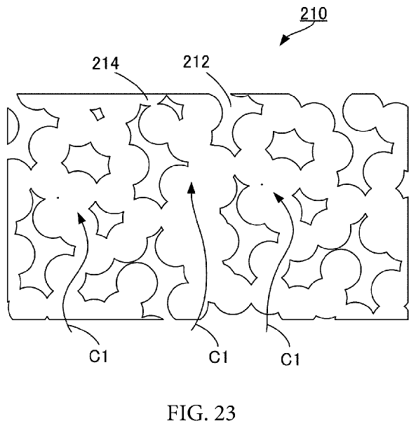

A sixth embodiment will be described focusing on portions differing from the first embodiment. As illustrated in FIG. 21, in a sponge 1E of the sixth embodiment, the slits 22 do not pass through the main body 10 in the vertical direction, and an intermediate portion between the upper surface 10a and the back surface 10b becomes a region where no slits 22 are formed. As a result, even if the slits 22 are formed, since the cell portions 14 (see FIG. 5) are not completely severed in the vertical direction, the liquid foundation is able to be effectively impregnated in the horizontal direction.

The cosmetic container of the present invention is not limited to the above embodiments, and various modifications can be made without departing from the gist of the present invention.

### REFERENCE SIGNS LIST

1, 1A, 1B, 1C, 1D, 1E, 1F Sponge
10 Main body
12 Resin portion
14 Cell portion
20, 30, 21, 22 Slit
20A First portion
20B Second portion
40 Cosmetic container
46 Puff

1. A liquid material impregnation member for impregnating and holding a cosmetic composition, comprising
a foamed plastic body formed with open cells; wherein
the main body has an outer shape defined by a side surface, and an upper surface and a back surface having an area greater than the side surface;
in the main body, a plurality of slits are formed so as to be separate from each other;
a plurality of the slits are distributed throughout the main body and are formed so as to extend in a direction connecting the upper surface and the back surface;
the distance between the plurality of the slits formed in the central portion of the main body is smaller than the distance between the plurality of the slits formed outside of the central portion;
the slits serve as a pathway for the cosmetic composition to reach the open cells; and
the open cells are configured to function primarily as a holding portion of the cosmetic composition.

2. The liquid substance impregnating member according to claim 1, wherein
an area ratio (Sc/S1), which is a ratio of the total area (Sc) of slit surface which is the inner surface of the main body formed by the plurality of the slits, to the area (S1) of the upper surface or back surface is configured to be within a predetermined numerical range of 1 or more.

3. The liquid substance impregnating member according to claim 1, wherein the slits are formed so as to pass through the main body in a vertical direction.

4. The liquid substance impregnating member according to claim 1, wherein the slits are formed so as to be exposed on either of the upper surface or the back surface, but not exposed on the other surface.

5. The liquid substance impregnating member according to claim 1, wherein the slits are exposed on both the upper surface and the back surface and are formed so as not to pass through the main body.

6. The liquid substance impregnating member according to claim 1, wherein
a portion of the slits passes through the main body in a vertical direction; and
the other portion of the slits is exposed on either of the upper surface or the back surface, but not exposed on the other surface.

7. The liquid material impregnating member according to claim 1, wherein
the number of the slits is defined as a number within a predetermined range with respect to the area of the upper surface or the back surface.

8. The liquid material impregnating member according to claim 1, wherein
the length of the slits is defined as a length within a predetermined range.

9. The liquid material impregnating member according to claim 1, wherein
the slits are formed in a cross shape.

10. The liquid material impregnating member according to claim 1, wherein
the slits are formed based on a positional reference of a plurality of virtual concentric circles centered on a predetermined position in the central portion of the main body; and
the radius of a virtual concentric circle one outside a specified virtual concentric circle of the virtual concentric circles is defined as differing only by a predetermined length from the radius of the specified virtual concentric circle of the virtual concentric circles.
